(19) United States
(12) Patent Application Publication
(10) Pub. No.: US 2020/0330377 A1
(43) Pub. Date: Oct. 22, 2020

(54) Biodegradable Polymer Based Implants for Ocular Drug Delivery

(71) Applicant: UNIVERISTY OF CINCINNATI, Cincinnati, OH (US)

(72) Inventors: Rupak Banerjee, Mason, OH (US); Soumyarwit Manna, Cincinnati, OH (US); James Augsburger, Cincinnati, OH (US)

(73) Assignee: University of Cincinnati, Cincinnati, OH (US)

(21) Appl. No.: 16/882,850

(22) Filed: May 26, 2020

Related U.S. Application Data
(63) Continuation of application No. 14/052,172, filed on Oct. 11, 2013, now abandoned.
(60) Provisional application No. 61/712,337, filed on Oct. 11, 2012.

(51) Int. Cl. A61K 9/00; A61K 31/519; A61K 45/06

(52) U.S. Cl. CPC A61K 9/0051; A61K 31/519; A61K 47/36; A61K 9/0092; A61K 45/06

(57) ABSTRACT

Novel sustained release biodegradable implants and methods of making and of using the same to treat ocular diseases are provided.

### CROSS REFERENCE TO RELATED APPLICATION

This application claims benefit to U.S. Provisional Application Ser. No. 61/712,337, filed Oct. 11, 2012, which is incorporated by reference herein in its entirety.

### TECHNICAL FIELD

The invention relates to sustained release biodegradable intraocular implants and methods of making and using the same for the treatment of ocular disorders.

### BACKGROUND

Known intravitreal implants are generally based on hydrophobic biodegradable polymers, for example lactic acid and glycolic acid based matrices such as poly-lactic acid (PLA), poly-glycolic acid (PGA), their copolymers and derivatives poly(lactic-co-glycolic) acid (PLGA). The degraded products of these polymers are metabolized to produce carbon dioxide and water. One limitation with the existing hydrophobic polymer matrices (PLA, PGA, and PLGA) is that they do not blend well with hydrophilic drugs, for example methotrexate. Another disadvantage of these hydrophobic matrices is that they degrade very slowly even after the drug has been released, resulting in local toxicity.

The known sustained release intravitreal implants which are also FDA approved include Retisert™ (Bausch & Lomb) and Ozurdex™ (Allergan). Retisert is a silicone-based disc shaped non-biodegradable implant comprising the cortico steroid fluocinolone acetonide approved to treat uveitis and diabetes macular edema over a period of 30 months. Ozurdex is a pellet shaped PLGA based implant that administers Dexamethasone and is approved to treat uveitis and diabetes macular edema over a period of 6 months. In these exemplary devices, the drug administered is hydrophobic in nature, which binds well with a hydrophobic polymer matrix reservoir made of PLGA or silicones. Since the drug is hydrophobic in nature, it exhibits a sustained release due to an inherent property of limited diffusivity in the vitreous medium of the eye.

The inventors are unaware of any devices similarly effective for sustained release of hydrophilic drugs in the intravitreal domain. Hence, the currently accepted routes of administration for desired hydrophilic agents is generally by intravitreal injection, which does not generally afford an opportunity for sustained-release. Treatments requiring long-term exposure to a therapeutic agent can be highly aversive to a patient.

As such, there remains a need for a sustained release biodegradable implant and methods of using the same that maintains the therapeutic dosage of hydrophilic drugs such as methotrexate, over a prolonged treatment time period, thereby improving the effectiveness and safety of treatment methods of various ocular diseases, including ocular diseases in the vitreoretinal domain such as primary intraocular lymphoma.

### SUMMARY

Accordingly, the present invention provides biodegradable intraocular implants that provide sustained release of hydrophilic therapeutic agents and methods of making and using the same to treat various ocular disorders. Specific embodiments are directed to sustained release biodegradable PLA coated chitosan-methotrexate implants, methods of making and using the same to treat various ocular diseases manifested in the vitreoretinal domain. According to a very specific embodiment, ocular diseases such as primary intraocular lymphoma may be effectively treated.

One embodiment is directed to a biodegradable intraocular implant adapted to provide sustained release of an effective amount of a therapeutic agent to an intraocular region of the eye. The implant is comprised of a swellable polymeric core comprising a hydrophilic therapeutic agent distributed throughout a hydrophilic polymer matrix at a concentration; a degradable hydrophobic polymer coating disposed about the surface of the swellable core, the coating being permeable to the therapeutic agent and the coating having a thickness, wherein upon implantation into the eye, the implant is effective to achieve sustained release of the therapeutic agent for a release duration.

Another embodiment is directed to a process for making a sustained release biodegradable intraocular implant. The process comprises the steps of: mixing a hydrophilic therapeutic agent with a hydrophilic polymer matrix; injecting the mixture into medical grade chemically inert flexible tubing; lyophilizing said tubing containing said mixture to obtain hydrophilic agent-hydrophilic polymer fibers; extracting said hydrophilic therapeutic agent-hydrophilic polymer fibers from the tubing; cutting the hydrophilic drug-hydrophilic polymer fibers into a desired implant length to form a swellable polymeric core; dip-coating the core into a hydrophobic coating solution, the hydrophobic coating solution having a concentration; and drying the coated core to yield a biodegradable sustained release intraocular implant having a degradable hydrophobic polymer coating disposed about a swellable polymeric core, the coating having a thickness and being permeable to the therapeutic agent.

According to another embodiment, a method of treating an ocular condition of an eye of a patient is provided. The method comprises placing a sustained release biodegradable intraocular implant into an intraocular region, the implant comprising a swellable polymeric core of hydrophilic therapeutic agent distributed throughout a hydrophilic polymeric matrix in a concentration, said core coated with a hydrophobic polymer permeable to the therapeutic agent, said coating having a thickness, wherein the therapeutic agent is delivered to the intraocular region through a combination of diffusion through the permeable membrane, swelling of the core, and degradation of the coating, for a release duration effective to treat the ocular condition.

### BRIEF DESCRIPTION OF THE DRAWINGS

FIG. 1 shows optical microscopy images depicting the dimensions and appearance of: 1A. a longitudinal view of PLA-coated implant; 1B. a longitudinal view of uncoated implant; 1C. a cross sectional view of PLA-coated implant showing PLA coating on the edge; 1D. a cross sectional view of uncoated implant. (Scale Bar=500 μm).

FIG. 2 shows Scanning Electron Microscopy images of a longitudinal view depicting the surface microstructure and morphology of: 2A. uncoated implant at 26×; 2B. uncoated implant at 80×; 2C. uncoated implant at 200×; 2D. coated implant at 26×; 2E. coated implant at 80×; 2F. coated implant at 200×.

FIG. 3 shows Scanning Electron Microscopy images of the cross-sectional view depicting the surface microstructure and morphology of: 3A. uncoated implant at 80×; 3B. uncoated implant at 200×; 3C. uncoated implant at 500×; 3D. PLA coated implant at 80×; 3E. PLA coated implant at 200×; 3F. PLA coated implant at 500×.

FIG. 4 shows the successful coating of PLA on the surface of the coated implant as determined by the Time of Flight-Secondary Ion Mass Spectroscopy spectra of PLA (MW 150,000) (blue), PLA coated 40% chitosan-methotrexate implant (red) and uncoated 40% chitosan-methotrexate implant (green).

FIG. 5 shows the characteristic DSC curve of a PLA coated implant showing the Tg around 50° C.

FIG. 6A shows the characteristic methotrexate UV-Vis Spectra for different concentrations. FIG. 6B shows the calibration curve for methotrexate peak at 258 nm. FIG. 6C shows the calibration curve of methotrexate peak at 302 nm. FIG. 6D shows the calibration curve for methotrexate peak at 372 nm.

FIG. 7A shows the release rate curves from uncoated chitosan-methotrexate implants with different drug loadings. FIG. 7B shows the release rate curves from uncoated chitosan-methotrexate implants with different drug loadings in the therapeutic window (shaded region). FIG. 7C shows the cumulative drug release profile from uncoated chitosan-methotrexate implants.

FIG. 8A shows the release rate curves from PLA coated chitosan-methotrexate implants with different drug loadings. FIG. 8B shows the release rate curves from PLA coated chitosan-methotrexate implants with different drug loadings in the therapeutic window (shaded region). indent FIG. 8C shows the cumulative drug release profile from PLA coated chitosan-methotrexate implants.

FIG. 9A shows the fitting of methotrexate release from the PLA coated chitosan-methotrexate implants using the Korsmeyer Peppas equation (for the first 60% of drug release). FIG. 9B shows the fitting of methotrexate release from the PLA coated chitosan-methotrexate implants using the first order equation (from the 10^{th} day to the end of therapeutic drug release).

### DETAILED DESCRIPTION

Particular details of various embodiments of the invention are set forth to illustrate certain aspects and not to limit the scope of the invention. It will be apparent to one of ordinary skill in the art that modifications and variations are possible without departing from the scope of the embodiments defined in the appended claims. More specifically, although some aspects of embodiments of the present invention may be identified herein as preferred or particularly advantageous, it is contemplated that the embodiments of the present invention are not necessarily limited to these preferred aspects.

Unless defined otherwise, all technical and scientific terms used herein have the same meaning as commonly understood by one of ordinary skill in the art to which the presently-disclosed subject matter belongs.

In certain embodiments, a biodegradable intraocular implant adapted to provide sustained release of an effective amount of a therapeutic agent to an intraocular region of the eye is provided. “Intraocular implant” refers to a device or element that is sized, structured, or otherwise configured to be placed in an eye and that can release a therapeutic agent over a sustained period of time, including days, weeks, and even months. Intraocular implants can be placed in an eye without disrupting vision of the eye, and intraocular implants are generally biocompatible with physiological conditions of the eye and do not cause adverse side effects.

The disclosed implants are comprised of a swellable polymeric core comprising a hydrophilic therapeutic agent distributed throughout a hydrophilic polymer matrix at a concentration. In some embodiments, the swellable polymeric core comprises hydrophilic therapeutic agent-hydrophilic polymer fibers. The hydrophilic therapeutic agent may be homogenously distributed throughout the core of the implant. As used herein, a “hydrophilic therapeutic agent” refers to a portion of the intraocular implant comprising one or more hydrophilic substances used to treat a medical condition of the eye. The hydrophilic therapeutic agent may be any hydrophilic pharmacologically active agent, either alone or in combination, for which sustained and controlled release is desirable and may be employed. The hydrophilic therapeutic agents are typically opthalmically acceptable, and are provided in a form that does not cause adverse reactions when the implant is placed into the eye. In some embodiments, the hydrophilic therapeutic agent is selected from the group comprising of methotrexate, carboplatin, cisplatin, cladribine, cyclophosphamide, cytarabine, doxorubicin, floxuridine, fluorouracil, gemcitabine hydrochloride, hydroxyurea, ifosfamide, mechlorethamine hydrochloride, mitomycin, topotecan, and combinations thereof. In certain embodiments, the hydrophilic therapeutic agent is methotrexate. In some embodiments, the swellable polymeric core comprises 10%, 25%, or 40% by weight hydrophilic agent.

The rate of release and the release duration of the hydrophilic therapeutic agent can be controlled by the loading concentration of the hydrophilic therapeutic agent, the weight and size of the hydrophilic therapeutic agent, and the solubility of the hydrophilic therapeutic agent.

The term “hydrophilic polymer matrix” refers to a hydrophilic polymer or polymers which degrade in vivo, and wherein the erosion of the hydrophilic polymer or polymers over time occurs concurrent with the subsequent release of the hydrophilic therapeutic agent. The term includes hydrophilic polymers which act to release the hydrophilic therapeutic agent through polymer swelling. A hydrophilic polymer matrix may be a homopolymer, copolymer, or a polymer comprising more than two different polymeric units. In some embodiments, the hydrophilic polymer matrix is selected from the group comprising chitosan, hydroxyethylcellulose, hydroxypropylmethylcellulose, and hydroxyproplycellulose, and mixtures thereof. In certain embodiments, the hydrophilic polymer matrix comprises chitosan.

The rate of release and release duration of the hydrophilic therapeutic agent will be controlled in part by the rate of transport through the hydrophilic polymeric matrix of the implant, and thus will be affected by the rate of swelling of different hydrophilic polymers and combinations thereof upon water absorption so as to make the hydrophilic polymer matrix more permeable to the hydrophilic therapeutic agent. Thus, the rate of release and the release duration of the hydrophilic therapeutic agent from the hydrophilic polymer matrix can be controlled by the use of different hydrophilic polymers and combinations thereof. The selection of a particular hydrophilic polymer matrix composition will vary depending on the desired release kinetics of the hydrophilic therapeutic agent and compatibility with the therapeutic agent, as well as the nature of the disease being treated, the implantation site, and the like.

A degradable hydrophobic polymer coating is disposed about the surface of the swellable core, with the coating having a thickness and being permeable to the therapeutic agent. As used herein, a “hydrophobic polymer coating” refers to a hydrophobic polymer or polymers which degrade in vivo and refers to a portion of the intraocular implant that is effective to provide a sustained release of the hydrophilic therapeutic agents of the implant. The erosion of the hydrophobic polymer or polymers over time occurs concurrent with the subsequent release of the hydrophilic therapeutic agent. Besides imparting hydrophobicity to the surface of the implant, the hydrophobic polymer coating prevents the entry of water into the hydrophilic polymer matrix, thereby reducing the rate of swelling of the hydrophilic polymer matrix and subsequent hydrophilic therapeutic agent release. A hydrophobic polymer coating may be a coating covering a core region of the implant that comprises a hydrophilic therapeutic agent distributed throughout a hydrophilic polymer matrix. A hydrophobic polymer coating may be a homopolymer, copolymer, or a polymer comprising more than two different polymeric units.

The rate of release and release duration of the hydrophilic therapeutic agent can be effected by the degradation and erosion rate of the hydrophobic polymer coating. Thus, the rate of release and release duration of the hydrophilic therapeutic agent can be controlled by the use of different hydrophobic polymers and mixtures thereof. Additionally, the thickness of the hydrophobic polymer coating can be used to control the rate of release and release duration of the hydrophilic therapeutic agent, and in some embodiments the release duration is inversely proportional to the hydrophobic polymer coating thickness. The thickness of the hydrophobic polymer coating can be controlled by several factors, including the molecular weight of the coating polymer or polymers and the concentration of the coating solution used to make the hydrophobic coating. Thus, the selection of a particular hydrophobic polymer coating composition will vary depending on the desired release kinetics of the hydrophilic therapeutic agent and compatibility with the therapeutic agent, as well as the nature of the disease being treated, the implantation site, and the like.

In one specific embodiment, a hydrophobic PLA coating is 100 μm thick. Additionally, different hydrophobic polymers can be selected for appropriate hydrophobic surface properties, time dependent degradation properties (biodegradation) and biocompatibility. In some embodiments the hydrophobic polymer coating is selected form the group comprising polylactic acid, poly(lactic-co-glycolic) acid, polyanhydride, polycaprolactone, and polyorthoesters. In other embodiments, the hydrophobic polymer coating comprises polylactic acid.

Upon implantation into the eye, the implant is effective to achieve sustained release of the therapeutic agent for a release duration. As mentioned previously, the rate of release and the release duration of the therapeutic hydrophilic agent are controlled by a variety of factors, including but not limited to, the loading concentration of the hydrophilic therapeutic agent, the size of the hydrophilic therapeutic agent, the solubility of the hydrophilic therapeutic agent, the use of different hydrophilic polymers and combinations thereof, the rate of diffusion of the hydrophilic therapeutic agent through the hydrophilic polymers, the rate of swelling of the hydrophilic polymers, the degradation and erosion rate of the hydrophobic polymer coating, the thickness of the hydrophobic coating, and the size and shape of the implant. In some embodiments, the release duration is inversely proportional to the hydrophobic polymer coating thickness. In certain embodiments, the release duration is about one month, while in other embodiments the release duration is about 8-10 weeks. In certain specific embodiments, the rate of release of the hydrophilic therapeutic agent methotrexate is 0.2-2.0 μg/day.

The therapeutic agent release rate data of certain specific embodiments of a PLA coated chitosan- methotrexate implants of the present invention were fitted to pharmacokinetic models to interpret the therapeutic agent diffusion kinetics. Therapeutic agent release data of all methotrexate loadings (10%, 25%, and 40% by weight of the swellable polymeric core) of the coated implants were fitted to zero order equation, first order equation, Higuchi model and Korsmeyer-Peppas model in order to analyze the mechanism of drug release and diffusion kinetics. The fitting of each model is evaluated based on correlation coefficient (R²) values. The R² values of each model fitting are reported in Table 1.

TABLE 1
In vitro release kinetic values of methotrexate from PLA coated
chitosan-methotrexate implants of different drug loadings.

| Methotrexate | First 8 days | | | 10^{th} day - end of therapeutic release | |
|---|---|---|---|---|---|
| loading | (60% drug release during initial burst) | | | (drug release in therapeutic window) | |
| w/w % | Korsmeyer Peppas | | Zero Order | First Order* | Higuchi |
| (N = 3) | R² | n | R² | R² | R² |
| 10% | 0.99 | 1.22 | 0.98 | 0.83 | 0.99 |
| 25% | 0.99 | 1.24 | 0.99 | 0.94 | 0.94 |
| 40% | 0.99 | 1.24 | 0.99 | 0.98 | 0.93 |

*The half-life (t_{1/2}) obtained from the first order kinetics for the whole range of drug release is ~10 days

The Korsmeyer-Peppas model provides an insight into the type of drug release mechanism taking place from swellable polymeric devices. The ‘n’ of the Korsmeyer Peppas model is estimated from the linear regression fit of the logarithmic release rate data. n>1 suggests super case II transport relaxational release and also indicates zero order kinetics. The generic equation for the Korsmeyer Peppas model is as follows:

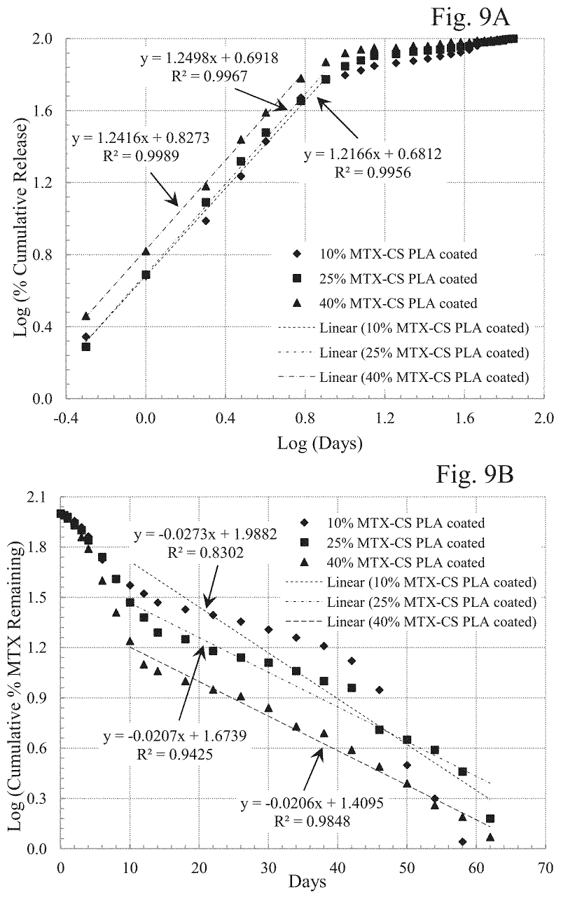

F=(Mₜ/M₀)=Kₖₚtⁿ (1)

where M₀ is the initial amount of drug, Mₜ is the amount of drug released in time t, F is the fraction of drug released at time t, Kₖₚ is the Korsmeyer Peppas release constant and n is estimated from linear regression of log F versus log t; n suggests the type of diffusion. Consistent R² values ˜0.99 and ‘n’ values ˜1.2 were obtained by fitting the first 60% of drug release rate data to the Korsmeyer Peppas model (FIG. 9A), suggesting that the first 60% of the drug release is influenced by swelling and relaxation phenomena of the polymer matrix. The 60% of the drug release takes place in the first 8 days out of the total drug release duration. If the whole range of drug release data is it to the Korsmeyer Peppas model, then the R² values reduce to 0.82-0.89 and the ‘n’ values vary between 0.62-0.73.

The zero order release equation represents a process when the release rate of the drug is independent of the concentration of the drug in the system and the generic equation for the zero order equation is as follows:

Mₜ=M₀+Kₒt (2)

where M₀ is the initial amount of drug, Mₜ is the amount of drug released in time t, K₀ is the zero order release constant. The range of R² values is between 0.02 and 0.49 when the whole range of drug release data is fitted to the zero order equation. R² values improve to ˜0.9 when the initial 60% drug release data is fitted to the zero order equation (Table 1). Therefore the drug release from the coated implants follows zero order equation for the first 60% of the drug release.

The first order release equation represents a system where the release rate of the drug is dependent on the concentration of the drug in the system and the generic equation for the first order equation is as follows:

log Mₜ=log M₀+K₁(t/2.303) (3)

where M₀ is the initial amount of drug, Mₜ is the amount of drug released in time t and K₁ is the first order release constant. The R² values are ˜0.9 when the whole range of drug release data is fitted to the first order equation. However, by fitting the drug release data to the first order equation from the 10^{th} day to the end of the drug release (˜60 days) provides the R² values of 0.83, 0.94 and 0.98 for 10%, 25% and 40% coated implants respectively (FIG. 9B). This implies the drug release rate from the coated implants in the therapeutic window, after the 10^{th} day (post-initial burst), is primarily governed by first order kinetics and is dependent on the concentration of the drug in the coated implants. The half life (t_{1/2}) of methotrexate release from an intravitreal injection is reported to be ˜14.3 hours, whereas the t_{1/2} of methotrexate release from the coated implants for the whole range of data is ˜240 hours (10 days) (Table 1).

The Higuchi release equation predicts that the drug release is caused primarily by diffusion mechanism and the generic equation for the Higuchi model is as follows:

Mₜ=K_{H}t^{1/2} (4)

where Mₜ is the amount of drug released in time t and K_{H} is the Higuchi constant. The range of R² values is between 0.7 and 0.91 when the whole range of drug release data is fitted to the Higuchi model. However, fitting the drug release data to the Higuchi model from the 10^{th} day to the end of drug release (˜60 days) provides the R² values of 0.99, 0.94 and 0.93 for 10%, 25% and 40% coated implants respectively (Table 1). This implies the drug release from the coated implants, after the 10^{th} day (post-initial burst), is primarily governed by diffusion kinetics.

Therefore, it can be concluded that the drug release mechanism primarily follows i) Korsmeyer Peppas model, and zero order model for the first ˜8 days where the initial burst takes place and 60% of the drug is released due to swelling of the polymer matrix; and ii) first order and Higuchi model from the 10^{th} day till the end of drug release signifying the drug release mechanism being concentration dependent and is primarily caused by diffusion mechanism, as shown in FIG. 9B.

In some embodiments of the presently-disclosed subject matter, a process for making a sustained release biodegradable intraocular implant is provided. In certain embodiments the process comprises mixing a hydrophilic therapeutic agent with a hydrophilic polymer matrix and injecting the mixture into medical grade chemically inert flexible tubing. The tubing containing said mixture is lyophilized to obtain hydrophilic agent-hydrophilic polymer fibers, and the hydrophilic therapeutic agent-hydrophilic polymer fibers are extracted from the tubing. The hydrophilic drug-hydrophilic polymer fibers are then cut into a desired implant length to form a swellable polymeric core. The core is then dip-coated into a hydrophobic coating solution having a certain concentration. The coated core is then dried to yield a biodegradable sustained release intraocular implant having a degradable hydrophobic polymer coating disposed about a swellable polymeric core, the coating having a thickness and being permeable to the therapeutic agent.

In some embodiments of a process for making a sustained release biodegradable intraocular implant, the hydrophobic coating solution comprises a polymer selected from the group consisting of polylactic acid, poly(lactic-co-glycolic) acid, polyanhydride, polycaprolactone, and polyorthoester. In certain embodiments, the hydrophobic coating solution concentration is proportional to the thickness of the hydrophobic polymer coating. In other embodiments, the hydrophobic coating solution concentration is 40 mg/ml.

In some embodiments of a process for making a sustained release biodegradable intraocular implant, the hydrophilic polymer matrix is selected from the group comprising chitosan, hydroxyethylcellulose, hydroxypropylmethylcellulose, and hydroxyproplycellulose. In certain embodiments, the hydrophilic therapeutic agent is selected from the group comprising methotrexate, carboplatin, cisplatin, cladribine, cyclophosphamide, cytarabine, doxorubicin, floxuridine, fluorouracil, gemcitabine hydrochloride, hydroxyurea, ifosfamide, mechlorethamine hydrochloride, mitomycin, topotecan, and combinations thereof. In other embodiments, the swellable polymeric core comprises 10%, 25%, or 40% by weight hydrophilic therapeutic agent.

For certain specific embodiments of polylactic acid (PLA) coated chitosan-methotrexate implants of the present invention, the PLA coating is about 100 μM thick and the length and diameter of the PLA coated implant are 4.2±0.03 mm and 0.9±0.04 mm, respectively.

In another embodiment of the presently-disclosed subject matter, a method of treating an ocular condition of an eye of a patient is provided. The term “treating” or “treat” as used herein, refers to the level or amount of agent required to treat an ocular condition, or reduce or prevent ocular injury or damage without causing significant adverse side effects to the eye or region of the eye. As used herein, an “ocular condition” is a disease or ailment which affects or involves the eye or one or more regions of the eye. In some embodiments, the ocular condition is selected from the group consisting of intraocular lymphoma, primary central nervous system lymphoma, primary vitreo-retinal lymphoma, proliferative vitreo-retinopathy, uveitis, and retinal detachment, while in certain embodiments the ocular condition is intraocular lymphoma.

In some embodiments of a method of treating an ocular condition of an eye of a patient, a sustained release biodegradable intraocular implant is placed into an intraocular region of the patient. The implant comprises a swellable polymeric core of hydrophilic therapeutic agent distributed throughout a hydrophilic polymeric matrix in a concentration. In some embodiments, the swellable polymeric core comprises hydrophilic therapeutic agent-hydrophilic polymer fibers. The core is coated with a hydrophobic polymer permeable to the therapeutic agent, with the coating having a thickness. The therapeutic agent is delivered to the intraocular region through a combination of, but not limited to, diffusion through the permeable hydrophobic polymer coating, swelling of the core, and degradation of the hydrophobic polymer coating, for a release duration effective to treat the ocular condition.

In certain embodiments of a method of treating an ocular condition of an eye of a patient, the swellable polymeric core comprises hydrophilic therapeutic agent-hydrophilic polymer fibers. In other embodiments the hydrophobic polymer coating is selected form the group comprising polylactic acid, poly(lactic-co-glycolic) acid, polyanhydride, polycaprolactone, and polyorthoesters. In other embodiments, the hydrophobic polymer coating comprises polylactic acid. In some embodiments, the hydrophilic polymer matrix is selected from the group comprising chitosan, hydroxyethylcellulose, hydroxypropylmethylcellulose, and hydroxyproplycellulose, and mixtures thereof. In certain embodiments, the hydrophilic polymer matrix comprises chitosan.

In additional embodiments of a method of treating an ocular condition of an eye of a patient, the hydrophilic therapeutic agent is selected from the group comprising methotrexate, carboplatin, cisplatin, cladribine, cyclophosphamide, cytarabine, doxorubicin, floxuridine, fluorouracil, gemcitabine hydrochloride, hydroxyurea, ifosfamide, mechlorethamine hydrochloride, mitomycin, topotecan, and combinations thereof. In certain embodiments, the therapeutic agent is methotrexate. In other embodiments, the swellable polymeric core comprises 10%, 25%, or 40% by weight hydrophilic therapeutic agent.

In some embodiments of a method of treating an ocular condition of an eye of a patient, the release duration is inversely proportional to the hydrophobic polymer coating thickness. In certain embodiments, the release duration is about one month, while in other embodiments the release duration is about 8-10 weeks.

### EXAMPLES

The following examples are given by way of illustration and are in no way intended to limit the scope of the claims of the present invention.

### Example 1

This example illustrates particular embodiments of the process for making sustained release biodegradable intraocular implants of the present disclosure.

### Fabrication of the Implant

Methotrexate (MP Biomedical) is mixed with low molecular weight chitosan (M.W 50,000-190,000 and DA %>75%) (Sigma Aldrich) in dilute HCl to make different mixtures of 10%, 25% and 40% w/w drug loadings. These mixtures are then injected into Tygon® tubing ( 1/16 in I.D). The tubes containing the mixture are lyophilized at a temperature below −40° C. and pressure below 1200 mTorr for 2 hours (Millrock BT48A, Millrock Technology) to obtain chitosan-methotrexate fibers. The chitosan-methotrexate fibers extracted from the Tygon® tubing are cut into desired implant lengths using a surgical knife under an optical microscope to ensure accurate dimensions of the implant.

DL-PLA (M.W 150,000) (Lactel Biodegradable Polymers) is mixed in Dicholoromethane (Fisher Sci.) to synthesize a 40 mg/ml coating solution. The chitosan-methotrexate implants are then dip coated in the PLA coating solution for a hydrophobic surface coating. The dip coating protocol is carried out on both longitudinal directions of the implant to ensure uniform coating on the surface and on two ends of the implant. Each implant is dipped in the PLA solution for 5 sec and dried at room temperature for 2 min. This process is carried out 3 times in each direction, longitudinally. Subsequently, the implants are dried overnight at room temperature in dark conditions. After initial drying, the implants are vacuum dried overnight at 45° C. to evaporate the dichloromethane from the implant.

### Example 2

### Implant Characterization

This example illustrates the appearance, dimensions, microstructure morphology, and hydrophobicity of the PLA coating of certain embodiments of the sustained release biodegradable intraocular implants of the present disclosure. Optical microscopy and SEM techniques were utilized to assess the implant's material properties, including appearance, dimensions and microstructure morphology. Hydrophobicity of the PLA coating is evaluated using Time of Flight-Secondary Ion Mass Spectroscopy (ToF-SIMS) and Differential Scanning Calorimetry (DSC) studies.

### Dimensions and Morphology

Optical Microscopy (Keyence Digital Microscope, VHX-600) is used to assess the implant's dimensions and appearance. Scanning Electron Microscopy (SEM) (FEI XL 30-FEG, FEI) is used to assess the microstructure and morphology using an accelerating voltage of 15 KV. The implant samples are sputter coated prior to the SEM analysis in Argon plasma using an Au—Pd target for 1 min to cause them to be conductive.

A summary of the implant dimensions is provided in Table 2. For implant samples (n=9; 3 samples and 3 readings per sample), the dimensions of the uncoated type and the PLA coated type are measured using an optical microscope. The length and cross-sectional diameter of the uncoated implant are 4±0.04 mm and 0.7±0.03 mm, respectively. The length and cross-sectional diameter of the PLA coated implant are 4.2±0.03 mm and 0.9±0.04 mm, respectively.

TABLE 2
Summary of implant dimensions

Dimensions (mm)

| Implant | Length | Cross sectional diameter |
|---|---|---|
| surface | (Mean ± SD) | (Mean ± SD) |
| PLA Coated | 4.2 ± 0.03 | 0.9 ± 0.04 |
| Uncoated | 4.0 ± 0.04 | 0.7 ± 0.03 |

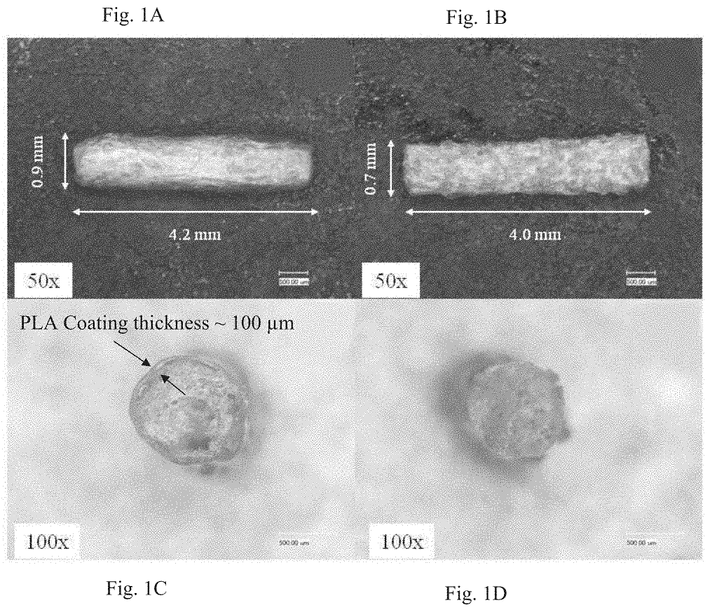

The optical microscopy images of surfaces of the PLA coated and the uncoated implants are shown in FIGS. 1A and 1B respectively. Comparing FIGS. 1A and 1B, it can be seen that the surface of the PLA coated implant is relatively smoother and more uniform compared to that of the uncoated implant. The optical microscopy images of the cross-sectional view of the PLA coated and uncoated implants are shown in FIG. 1C and FIG. 1D respectively. A 100 μm PLA coating is present in the PLA coated implant in FIG. 1C which is absent in the uncoated implant in FIG. 1D. The implants are a yellow color signifying uniform distribution of methotrexate throughout the chitosan polymer matrix. Thus, optical microscopy images reveal uniform coating of PLA on the surface of the PLA coated implants.

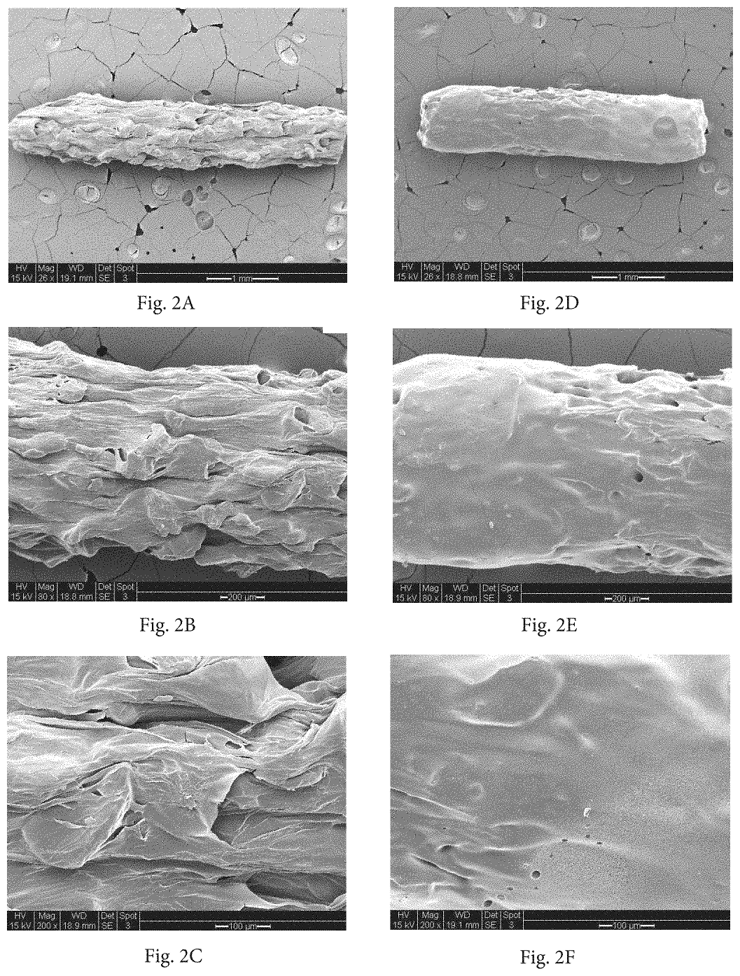

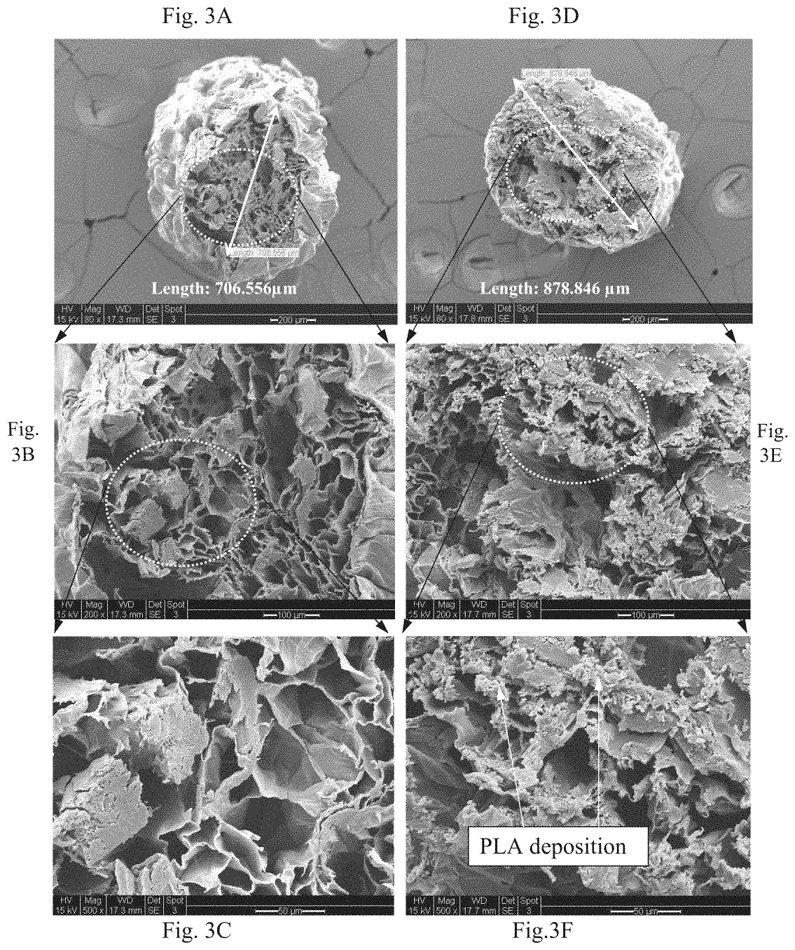

SEM images showing the longitudinal view of the surface of the uncoated and PLA coated implants are shown in FIG. 2. From the SEM images, the porous and irregular chitosan surface of the uncoated implant can be seen. By coating the implants with PLA, the porous surface gets filled up with PLA and results in a smoother non-porous surface as shown in the SEM images of the coated implant. SEM images of the cross section of the uncoated and the PLA coated implant are shown in FIG. 3. The cross-sectional diameter of the uncoated (0.706 mm) and the PLA coated (0.878) implants are shown in FIGS. 3A and 3D respectively. They are consistent (˜2.4% difference) with the results of optical microscopy as shown in FIG. 1. In FIGS. 3B and 3C, the porous internal chitosan matrix of the uncoated implant is shown. In FIGS. 3D and 3E, it is visible that the PLA deposition takes place in the internal voids of the coated implant resulting in a denser internal matrix with reduced porosity. The internal deposition of PLA also plays an important role in the reduction of swelling of the chitosan matrix and restricting the methotrexate release.

### Hydrophobic Modification of the Coated Implant Surface

ToF-SIMS is used to assess the hydrophobic modification of the implant's surface. ToF-SIMS is performed using a ToF-SIMS IV instrument (IONTOF Inc.). Secondary ions are produced from a Ga+ primary ion source at 15 KV accelerating voltage and 1.5 pA current raster over a 200 μm by 200 μm area of the sample. The secondary ions produced are analyzed in high-current bunched mode with analyzer energy of 2 KV. The ion peaks are assigned using SurfaceLab 6 software (IONTOF Inc.). DSC is used to measure thermal properties of the implants at physiological temperature ˜38° C. DSC is performed at the heating rate of 10° C./min. (DSC6200, Seiko Instruments Inc.).

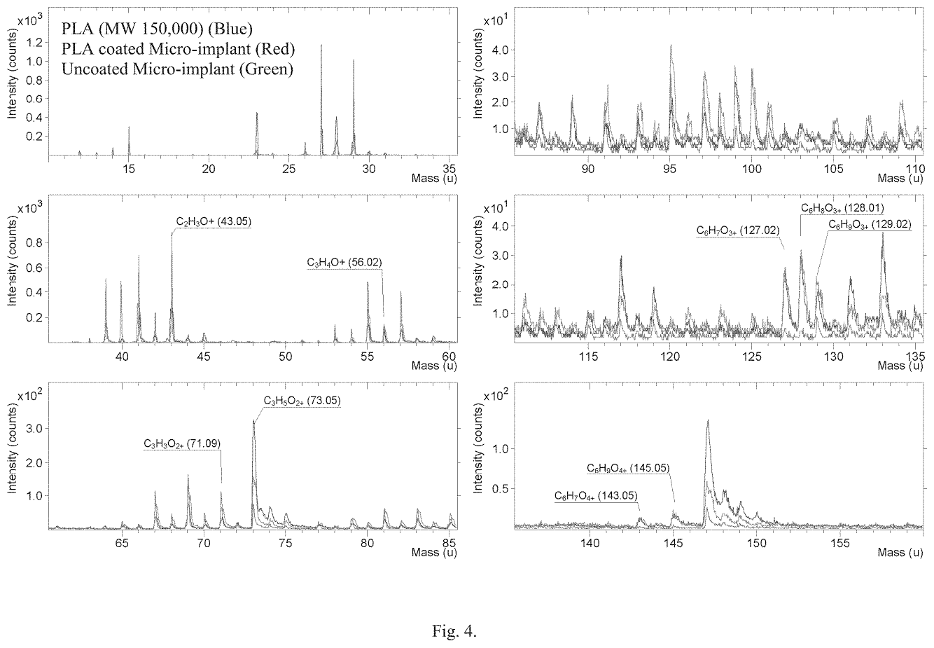

ToF-SIMS spectra of PLA (MW 150,000) (blue), PLA coated 40% chitosan-methotrexate implant surface (red) and uncoated 40% chitosan-methotrexate implant surface (green) are reported in FIG. 4. FIG. 4 shows the characteristic peaks (blue color) of pure PLA mass fragments (43 [C₂H₃O⁺], 56 [C₃H₄O⁺], 71 [C₃H₃O₂⁺], 73 [C₃H₅O₂⁺], 127 [C₆H₇O₃⁺], 128 [C₆H₈O₃⁺], 129 [C₆H₉O₃⁺], 143 [C₆H₇O₄⁺] and 145 [C₆H₉O₄⁺]) match with that of the PLA coated implant (red color) with similar intensities. The characteristic peaks of pure PLA mass fragments (blue color) and PLA coated implant (red color) match with previous study (Mahoney, C. M. et al., 2004, “Depth profiling of 4-acetamindophenol-doped poly(lactic acid) films using cluster secondary ion mass Spectrometry,” analytical chemistry, 76(11), pp. 3199-3207).

The spectrum of the uncoated implants (green color) does not show the same characteristic peaks (56 [C₃H₄O⁺], 71 [C₃H₃O₂⁺], 73 [C₃H₅O₂⁺], 127 [C₆H₇O₃⁺], 128 [C₆H₈O₃⁺], 129 [C₆H₉O₃⁺], 143 [C₆H₇O₄⁺] and 145 [C₆H₉O₄⁺]) as that of pure PLA mass fragments (blue color) and PLA coated implant (red color). However, in the spectrum of uncoated implants (green color), there is a match with the spectra of pure PLA mass fragments (blue color) and PLA coated implant (red color) at mass fragment 43 [C₂H₃O⁺], but with a much higher relative intensity than the spectra of the pure PLA mass fragments (blue color) and PLA coated implant (red color). The higher relative intensity from the uncoated implants is probably due to the mass fragment 43 [C₂H₃O⁺] being generated from the chitosan and methotrexate present on the surface of the uncoated implants. Therefore, the spectra of FIG. 4 qualitatively confirms the successful coating of PLA on the surface of the coated implant.

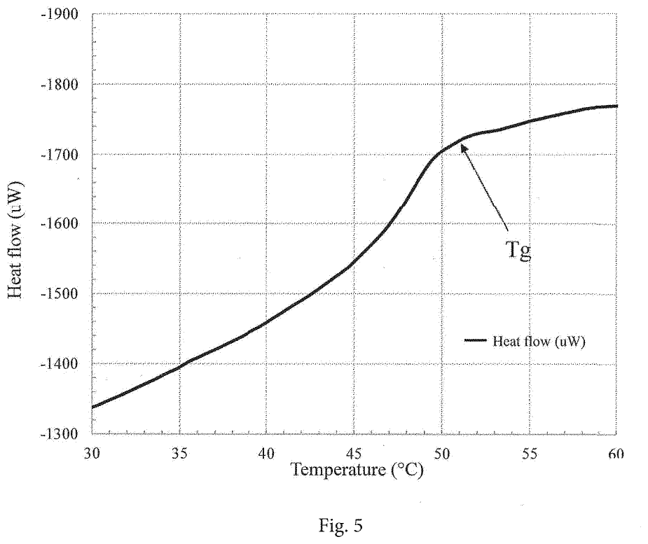

If the coating polymer PLA undergoes glass transition in the physiological conditions, then the PLA coating would soften, affecting the structural properties of the implant, thus leading to faster drug release. A DSC plot of one of the PLA coated implants is shown in FIG. 5. The glass transition temperature (Tg) is the point where the slope of the endotherm changes. The Tg values of PLA coated implants for different drug loadings are reported in Table 3. The Tg values range between 50-52° C., which are consistent with previous studies (Passerini, N., et al., 2001, “An investigation into the effects of residual water on the glass transition temperature of polylactide microspheres using modulated temperature DSC,” Journal of Controlled Release, 73(1), pp. 111-115). The DSC study confirms that the PLA coating will not degrade or experience glass transition or soften in the physiological temperature (˜38° C.) inside the intraocular domain.

TABLE 3
Summary of Tg of PLA coated implants of different drug loadings

| % Methotrexate loading | Tg (° C.) |
|---|---|
| (n = 4) | (Mean ± SD) |
| 10% | 50.2 ± 1.3 |
| 25% | 51.3 ± 1.1 |
| 40% | 51.9 ± 2.8 |

### Example 3

This example illustrates the rate of release and the release duration of the hydrophilic therapeutic agent from particular embodiments of the biodegradable intraocular implants of the present disclosure.

### Release Rate Studies

The implants are kept in vials containing 5m1 of phosphate buffered saline (PBS; pH 7.4). Each implant weighs ˜1 mg. The implants with 40% w/w methotrexate contain ˜400 μg of methotrexate, the implants with 25% w/w methotrexate contain ˜250 μg of methotrexate, and the implants with 10% w/w methotrexate contain ˜100 μg of methotrexate. The vials are slowly stirred in a water bath maintained at 38° C. 1 ml of release media sample (PBS) containing methotrexate is taken out at pre-determined time intervals. 1 ml of fresh PBS is added to maintain sink conditions. The concentration of methotrexate present in 1 ml of release media is assayed using a UV-Visible Spectrophotometer (Cary 50-Bio UV-Vis Spectrophotometer, Varian) at the characteristic methotrexate wavelengths (258,302 and 372 nm) (Kunou, N. et al., 2000, “Long-term sustained release of ganciclovir from biodegradable scleral implant for the treatment of cytomegalovirus retinitis,” Journal of Controlled Release, 68(2), pp. 263-271). The calibration of methotrexate absorbance in the UV-Visible Spectrophotometer is done using methotrexate standard concentrations in PBS. A calibration curve is derived from the absorbance readings obtained from the methotrexate standards and the molar absorbtivity of methotrexate is determined.

### Calibration of Methotrexate

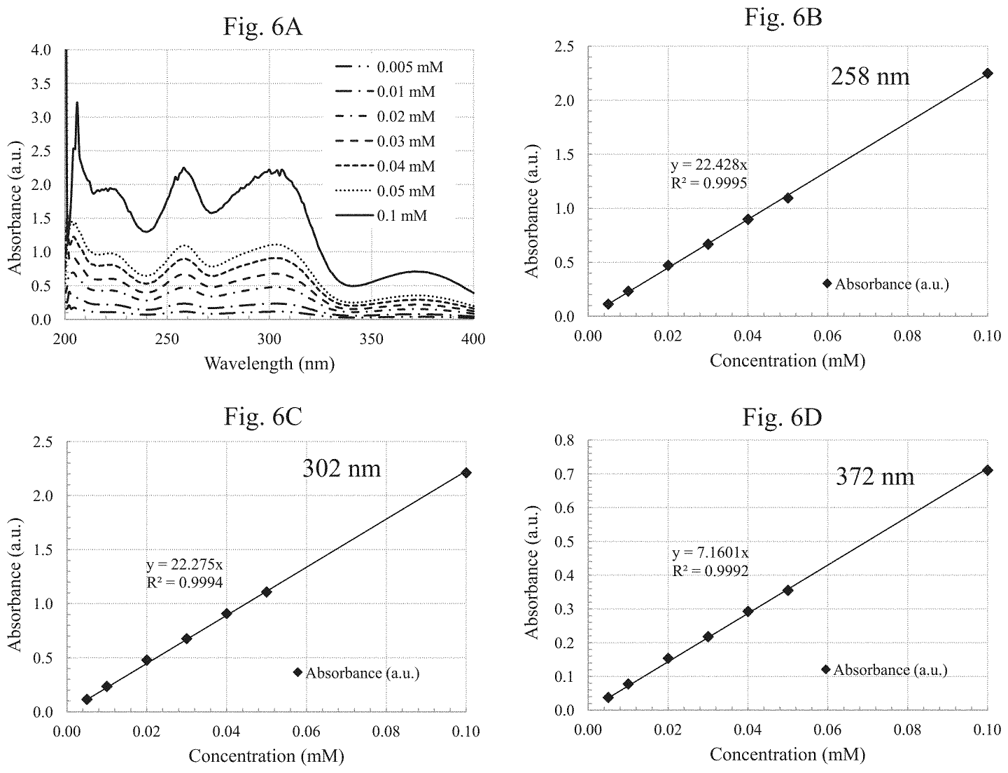

FIG. 6 describes the calibration procedure for methotrexate. Characteristic methotrexate spectra for different concentrations are shown in FIG. 6A. The characteristic methotrexate peaks are at 258 nm, 302 nm and 372 nm and the calibration curves for the 258 nm peak, 302 nm peak and 372 nm peak are shown in FIGS. 6B, 6C and 6D, respectively. The calibration curve of each peak is obtained by linear regression fitting of the UV-absorbance values for different methotrexate concentrations. The linear regression is based on terms of correlation coefficient (R²) values. The 258 nm peak of the methotrexate spectra is used for the release rate experiments as it provides a sharper deflection compared to the others.

### Release Rate Profiles

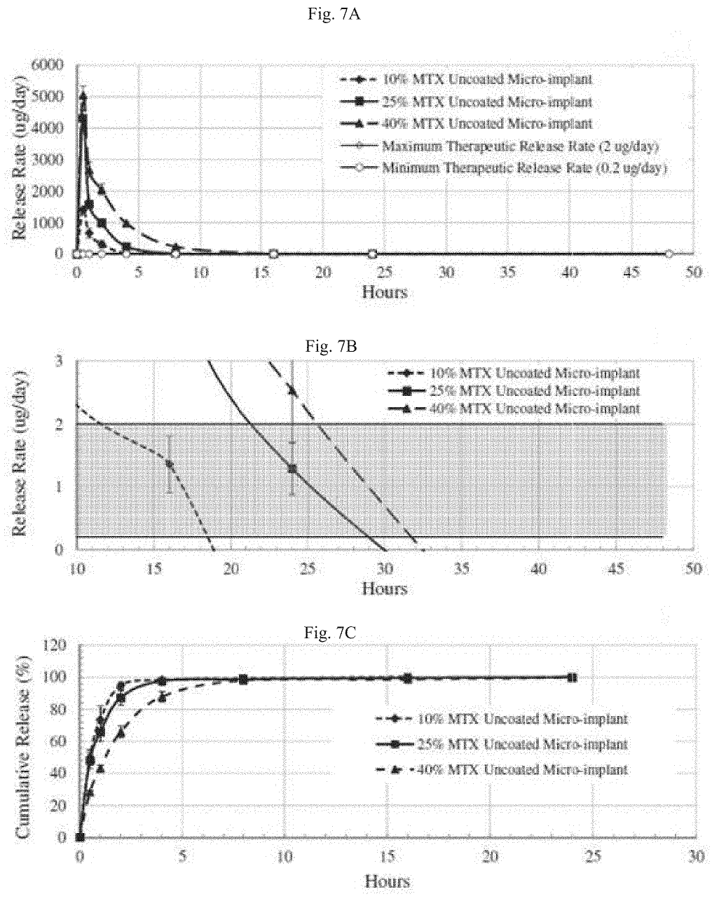

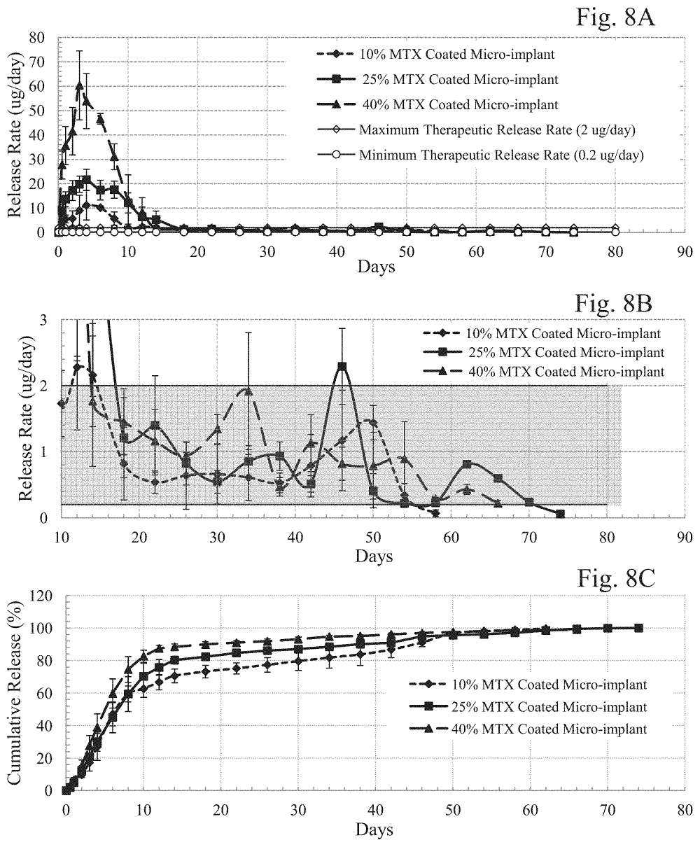

Release rate profiles of methotrexate from the uncoated implants are shown in FIG. 7A. FIG. 7B shows release rate profiles of methotrexate from the uncoated implants in the therapeutic window (0.2-2.0 μg/day). Cumulative release profiles of methotrexate from the uncoated implants are shown in FIG. 7C. Release rate profiles of methotrexate from the PLA coated implants are shown in FIG. 8A. FIG. 8B shows release rate profiles of methotrexate from the PLA-coated implants in the therapeutic window. Cumulative release profiles of methotrexate from the PLA-coated implants are shown in FIG. 8C. The mean profile of each type of drug loading is plotted along with the standard error. The summary of release rate characteristics for the uncoated and coated implants for different drug loadings is provided in Tables 4 and 5, respectively.

TABLE 4
Summary of release rate characteristics of uncoated chitosan-methotrexate implants (n = 3)

| | | | | | Start time of | Drug released | End time of |
|---|---|---|---|---|---|---|---|
| | | | Time of | | release rate | before therapeutic | release rate |
| Implant | Mean Release | Total | Peak | Pea Release | within | release rate | within |
| Drug | Rate ± Standard | Release | Release | Rate ± Standard | therapeutic | starts ± Standard | therapeutic |
| loading (w/w) | Error (μg/day) | Duration (hours) | Rate (hours) | Error (μg/day) | limits (hour) | Error (%) | limits (hour) |
| 10 | 88.9 ± 4.8 | 19 | 0.5 | 1413.5 ± 65.5 | ~12 | 50.0 ± 4.7 | ~19 |
| 25 | 188.0 ± 7.9 | 29 | 0.5 | 4314.4 ± 221.4 | ~22 | 98.8 ± 0.3 | ~29 |
| 40 | 372.6 ± 7.5 | 32 | 0.5 | 5041.2 ± 310.7 | ~25 | 98.7 ± 0.4 | ~32 |

TABLE 5
Summary of Release Rate Characteristics of PLA coated chitosan-methotrexate implants (n = 3)

| | | | | | Start time of | Drug released | End time of |
|---|---|---|---|---|---|---|---|
| | | | Time of | | release rate | before therapeutic | release rate |
| Implant | Mean Release | Total | Peak | Peak Release | within | release rate | within |
| Drug | Rate ± Standard | Release | Release | Rate ± Standard | therapeutic | starts ± Standard | therapeutic |
| loading (w/w) | Error (μg/day) | Duration (days) | Rate (days) | Error (μg/day) | limits (day) | Error (%) | limits (day) |
| 10 | 1.8 ± 0.4 | 58 | 4 | 11.2 ± 6.0 | 10 | 62.7 ± 5.3 | ~58 |
| 25 | 3.2 ± 0.1 | 74 | 4 | 21.6 ± 4.3 | 18 | 82.3 ± 1.5 | ~74 |
| 40 | 6.6 ± 0.3 | 66 | 3 | 60.4 ± 14.1 | 14 | 86.8 ± 1.0 | ~66 |

### Release Rate Study of the Uncoated Implants

The mean release rate of the uncoated chitosan-methotrexate implants is 88.9±4.8 μg/day, 188.0±7.9 μg/day and 372.6±7.5 μg/day for the 10%, 25% and 40% w/w drug loadings respectively as mentioned in Table 4. The total release duration is defined as the duration from the start of drug release till the time it remains in the therapeutic window. The total release duration for 10%, 25% and 40% w/w chitosan-methotrexate implants is 19, 29, and 32 hours respectively. The 10% w/w, 25% w/w and the 40% w/w implants remain in the therapeutic window between 12^{th} to 19^{th} hour, 22^{nd} to 29^{th} hour and 25^{th} to 32^{nd} hour respectively as shown in FIG. 7B.

### Release Rate Study of the PLA-Coated Implants

The mean release rate of the PLA coated chitosan-methotrexate implants is 1.8±0.4 μg/day, 3.2±0.1 μg/day and 6.6±0.3 μg/day for the 10%, 25% and 40% w/w drug loadings respectively as mentioned in Table 5. The total release duration for 10%, 25% and 40% w/w PLA coated chitosan-methotrexate implants are 58, 74 and 66 days, respectively.

For the 10% coated chitosan-methotrexate implant, there is an initial burst release on the 4^{th} day (FIG. 8A), then a small secondary burst between 10^{th} and 20^{th} day and a final burst near 50^{th} day (FIG. 8B). The 10% w/w coated implants exhibit a release rate in the therapeutic window from the 10^{th} day onward up to the 58^{th} day as shown in FIG. 8B.

For the 25% coated chitosan-methotrexate implant, an initial burst release is seen on the 3^{rd} day (FIG. 8A). Although there is no prominent secondary burst, there are a couple of bursts between 20^{th} and 40^{th} day, followed by a major burst between 40^{th} and 50^{th} day before a final burst around the 70^{th} day (FIG. 8B). The 25% w/w coated implants show a release rate in the therapeutic window from the 18^{th} day onward up to the 74^{th} day.

In the case of 40% coated chitosan-methotrexate implant, a significant initial burst release is noticed on the 3^{rd} day (FIG. 8A), and then a secondary burst is observed between 30^{th} and 40^{th} day (FIG. 8B). There is no prominent final burst noticed in the release profile of the 40% coated implant. The 40% w/w implants maintain the release rate in the therapeutic window from the 14^{th} day onward up to the 66^{th} day.

Thus, the data demonstrates that uncoated chitosan-methotrexate implants are able to administer the drug for approximately 1 day. This rapid release of methotrexate is expected because of the similar hydrophilic nature of both chitosan and methotrexate. However, the presently disclosed data demonstrates that a PLA coating imparts hydrophobicity to the surface of the chitosan-methotrexate implant, and that the PLA coated chitosan-methotrexate implants are able to administer the therapeutic release rate of 0.2-2.0 μg/day of methotrexate for more than 50 days.

The PLA coating plays an important role in sustained release administration of methotrexate and also influences the initial burst release or the peak release rate of methotrexate. Besides imparting hydrophobicity to the surface of the implant, the PLA coating prevents the entry of PBS into the chitosan matrix, thereby reducing the rate of swelling of the chitosan matrix and subsequent methotrexate release. The presently disclosed data further demonstrates that the sustained release of methotrexate from the PLA coated implants can also be attributed to the degradation rate of PLA coating. Thus, the presently disclosed data demonstrates that sustained release biodegradable intraocular implants that consist of a degradable hydrophobic polymer coating disposed about a swellable polymeric core comprising a hydrophilic therapeutic agent distributed throughout a hydrophilic polymer matrix at a concentration, can be used as an alternative to intravitreal injections for sustained release of the therapeutic agent and potentially better tolerance and improved efficacy in treating ocular diseases, including ocular diseases in the vitreo retinal domain, using minimally invasive surgical methods.

All documents cited are incorporated herein by reference; the citation of any document is not to be construed as an admission that it is prior art with respect to the present invention.

Having described embodiments of the present invention in detail, and by reference to specific embodiments thereof, it will be apparent that modifications and variations are possible without departing from the scope of the embodiments defined in the appended claims. More specifically, although some aspects of embodiments of the present invention are identified herein as preferred or particularly advantageous, it is contemplated that the embodiments of the present invention are not necessarily limited to these preferred aspects.

1-34. (canceled)

35. A cylindrical biodegradable intravitreal implant adapted to provide sustained release of an effective amount of a hydrophilic therapeutic agent to an intravitreal region of the eye, the implant comprising:
a lyophilized core comprising a porous chitosan matrix having a hydrophilic therapeutic agent distributed throughout at a concentration, wherein the core is comprises fibers;
a smooth, non-porous, degradable hydrophobic polymer coating that is uniformly disposed about the core fibers such that the hydrophobic polymer covers and fills the pores of the chitosan matrix uniformly, the uniform coating being permeable to the therapeutic agent while preventing entry of water to the core, and the uniform coating having a thickness such that upon implantation into the eye, the implant is effective to achieve sustained release of the therapeutic agent in the intravitreal region for a release duration and wherein,
the cylindrical biodegradable intravitreal implant is ophthalmically acceptable in a form that does not cause adverse reactions when the implant is placed in the eye.

36. The implant of claim 35, wherein the hydrophobic polymer coating is selected from the group comprising polylactic acid, poly(lactic-co-glycolic) acid, polyanhydride, polycaprolactone, and polyorthoesters.

37. The implant of claim 36, wherein the hydrophobic polymer coating comprises polylactic acid.

38. The implant of claim 35, wherein the hydrophilic therapeutic agent is selected from the group consisting of methotrexate, carboplatin, cisplatin, cladribine, cyclophosphamide, cytarabine, doxorubicin, floxuridine, fluorouracil, gemcitabine hydrochloride, hydroxyurea, ifosfamide, mechlorethamine hydrochloride, mitomycin, topotecan, and combinations thereof.

39. The implant of claim 38, wherein the hydrophilic therapeutic agent is methotrexate.

40. The implant of claim 35, wherein the core comprises 10%, 25%, or 40% by weight hydrophilic therapeutic agent.

41. The implant of claim 35, wherein the release duration is inversely proportional to the hydrophobic polymer coating thickness.

42. The implant of claim 35, wherein the release duration is at least about one month.

43. The implant of claim 35, wherein the release duration is at least about 8-10 weeks.

44. The implant of claim 35, wherein the core has a length of about 4 mm and a cross-sectional diameter of about 0.7 mm.

45. The implant of claim 35, wherein the coated core has a length of about 4.2 mm and a cross-sectional diameter of about 0.9 mm.

46. The implant of claim 37, wherein the hydrophobic polymer coating further comprises poly(lactic-co-glycolic) acid.

47. The implant of claim 37, wherein the hydrophobic polymer coating consists of polylactic acid.

48. The implant according to claim 39, wherein sustained release of the methotrexate is 0.2-2.0 μg/day for the release duration.

49. A process for making a sustained release biodegradable intravitreal implant, the process comprising:
mixing a hydrophilic therapeutic agent with a hydrophilic polymer matrix;
injecting the mixture into medical grade chemically inert flexible tubing;
lyophilizing the tubing containing the mixture to obtain hydrophilic therapeutic agent-hydrophilic polymer fibers;
extracting the hydrophilic therapeutic agent-hydrophilic polymer fibers from the tubing;
cutting the hydrophilic therapeutic agent-hydrophilic polymer fibers into a desired implant length to form a lyophilized core;
dip-coating the core into a hydrophobic coating solution, the hydrophobic coating solution having a concentration;
drying the coated core to yield a biodegradable sustained release intravitreal implant having a degradable hydrophobic polymer coating disposed about a core, the coating having a thickness and being permeable to the therapeutic agent.

50. The process of claim 49, wherein the hydrophobic coating solution comprises a polymer selected from the group consisting of polylactic acid, poly(lactic-co-glycolic) acid, polyanhydride, polycaprolactone, and polyorthoester.

51. The process of claim 49, wherein the hydrophobic coating solution concentration is proportional to the thickness of the hydrophobic polymer coating.

52. The process of claim 49, wherein the hydrophobic coating solution concentration is 40 mg/ml.

53. The process of claim 49, wherein the hydrophilic polymer matrix is selected from the group consisting of chitosan, hydroxyethylcellulose, hydroxypropylmethylcellulose, and hydroxyproplycellulose.

54. The process of claim 49, wherein the hydrophilic therapeutic agent is selected from the group consisting of methotrexate, carboplatin, cisplatin, cladribine, cyclophosphamide, cytarabine, doxorubicin, floxuridine, fluorouracil, gemcitabine hydrochloride, hydroxyurea, ifosfamide, mechlorethamine hydrochloride, mitomycin, topotecan, and combinations thereof.

55. The process of claim 49, wherein the core comprises 10%, 25%, or 40% by weight hydrophilic therapeutic agent.
